(19) United States
(12) Patent Application Publication
(10) Pub. No.: US 2008/0175810 A1
(43) Pub. Date: Jul. 24, 2008

(54) Topical compositions for cosmetic and pharmaceutical use

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 11/656,250

(22) Filed: Jan. 22, 2007

(51) Int. Cl. A61K 31/74; A61P 17/00

(52) U.S. Cl. 424/78.05; 424/78.03; 424/78.07

(57) ABSTRACT

This invention relates to topical compositions in the form of dispersion gels formed by dispersing into an aqueous gel phase a substantially homogenous organic solution containing at least one active ingredient and at least one organic polymer solubilized in an organic carrier. The organic carrier comprises at least one water-miscible organic solvent and at least one water-immiscible organic solvent. The polymer is selected to affect efficacy of the compositions. The compositions are essentially free of surface-active substances and have favorable galenic properties.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to compositions in the form of dispersion gels for topical delivery of cosmetic or pharmacologically active agents. This invention also relates to method of prophylactic or therapeutic treatment of a patient with the compositions.

### BACKGROUND OF THE INVENTION

The conventional preparation forms of topically applied cosmetic and pharmaceutical formulations consist mainly of solutions, gels, emulsions, ointments, and liposome preparations. These preparation forms generally have certain disadvantages. For example, gels and emulsions can cause skin irritation due to surface-active substances contained within them. Ointments have a fatty base, which the skin can only absorb slowly. They often make the area of application heavy and greasy. Liposome preparations are limited by their relatively low loading capacity.

More recently, a number of patent disclosures report preparations of topical compositions without use of surface-active substances. These compositions are dispersion gels formed by dispersing an organic phase into a gelled water phase. These compositions have favorable galenic properties compared to the conventional preparation forms.

For example, U.S. Pat. No. 5,035,890 discloses a clear gel preparation formulated by trapping active ingredient in a powdered carrier of cross-linked polymer and then dispersing the loaded carrier into a gelled water phase.

U.S. Pat. No. 5,833,951 discloses dispersion formulations of the oil-in-water type comprising non-polar oils, fats, and/or waxes as the oil phase and an aqueous gel phase, optionally the formulations may comprise active ingredients. However, there is no disclosure on how to affect efficacy of the compositions.

U.S. Pat. No. 5,894,019 discloses a composition comprising a liquid lipid phase containing active ingredient solubilized in the liquid lipid and a hydrous gel. The liquid lipid phase is dispersed in the hydrous gel without use of surface-active substances. There is no disclosure on how to affect efficacy of the compositions.

U.S. Pat. No. 6,054,138 discloses a composition comprising a hydrous gel and a lipid phase containing a consistency factor (e.g., waxes, triglycerides, or vegetable fats which have a melting point higher than 50.degree. C.) for improving stability of the composition. The lipid phase is dispersed in the hydrous gel.

It would be advantageous that efficacy of a topical composition can be modulated to provide a desired cosmetic benefit or treat a particular medical condition.

U.S. Pat. No. 6,211,250 discloses a homogenous liquid system capable of percutaneous delivery of active ingredient comprising an active ingredient, volatile solvent and a rate modulating carrier consisting of a mixture of hydrophobic and hydrophilic polymers. After evaporation of the volatile solvent, the active ingredient is trapped in a multi-polymer matrix formed by the hydrophobic and hydrophilic polymers. The active ingredient trapped in the polymer matrix offers enhanced efficacy. The polymer matrix, as a reservoir and release modulator, modulates delivery profile of the active ingredient.

However, it is well known that a large number of active ingredients (cosmetic or pharmacological) are hydrophobic molecules. Adequate water solubility at safe and pharmacologically effective level is a major obstacle for such active ingredients. High concentration of organic solvent or even pure phase of organic solvent is generally needed to prepare a substantially homogenous composition. High concentration of organic solvents can cause skin allergy and irritation.

Therefore, there is a need for topical composition containing organic polymers where the polymers are selected to affect efficacy of the composition and yet having favorable galenic properties and cosmetic elegance.

### SUMMARY OF THE INVENTION

One aspect of this invention is a topical composition in the form of a dispersion gel containing at least one active ingredient and at least one organic polymer solubilized in an organic carrier where the polymer is selected to affect efficacy of the composition.

Another aspect of this invention is a topical composition with favorable galenic properties and cosmetic elegance compared to conventional preparation forms and at mean time with affected efficacy.

A further aspect of this invention is a topical composition which is essentially free of surface-active substances, thereby minimizing potential of skin allergy and irritation.

Still other objects and advantages of the invention will, in part, be obvious and will, in part, be apparent from the following detailed description of the preferred embodiments.

### DETAILED DESCRIPTION OF THE PREFERRED EMBODIMENTS

A topical composition in the form of a dispersion gel in accordance with the present invention comprises an aqueous gel phase and an organic phase formed by dispersing into the aqueous gel phase a substantially homogenous organic solution containing active ingredients and organic polymers.

The term, ‘dispersion gel’, as used herein, describes a multi-phase system which has at least one organic phase dispersed in at least one aqueous gel phase. The organic phase is the internal phase (discontinuous phase). It is intermingled in the aqueous gel phase which is the external phase (continuous phase).

The organic solution comprises at least one solubilized active ingredient and at least one solubilized organic polymer in an organic carrier. The organic carrier consists essentially of a mixture of at least one water-miscible solvent and at least one water-immiscible solvent. The water-miscible and water-immiscible solvents are substantially miscible in each other, i.e., when the water-miscible and water-immiscible solvents are mixed, a substantially homogenous solution is obtained.

Water-miscible or water-immiscible organic solvents may be any cosmetically or pharmacologically acceptable solvents. A water-miscible organic solvent is generally defined as a solvent having greater than about 10% solubility in water. A water-immiscible organic solvent is generally defined as a solvent having less than about 10% solubility in water. Examples of the water-miscible solvents include, but not limited to: monohydric alcohols, polyhydric alcohols, polyalkenyl alcohols, N-alkyl-2-pyrrolidones, isosorbide type of solvents, dimethyl sulfoxide, dimethylformide, dimethyl acetamide, ethyl acetate, and acetone.

Examples of monohydric alcohols are ethanol, isopropanol, and 1-butanol. Examples of polyhydric alcohols are glycerin, propylene glycol, butylene glycol, and hexylene glycol. Examples of polyalkenyl alcohols are low molecular weight polyethylene glycols (PEGs), such as PEG 200, PEG 300, PEG 400, and PEG 600. Examples of N-alkyl-2-pyrrolidones are N-methyl-2-pyrrolidone, N-ethyl-2-pyrrolidone, and N-propyl-2-pyrrolidone. Examples of isosorbide type of solvents are dimethyl isosorbide and diethyl isosorbide.

Examples of the water-immiscible solvents are glycerides, esters of propylene glycol, vegetable oils, and esters of mid- to long-chain carboxylic acids (generally with 8 to 20 carbon atoms). Examples of glycerides are caprylic/capric triglyceride, caprylic/capric/linoleic triglyceride, and caprylic/capric/succinic triglyceride. Examples of esters of propylene glycol are propylene glycol dicaprylate and propylene glycol dicaprate. Examples of vegetable oils are castor oil, soy bean oil, canola oil, and sawflower oil. Examples of esters of the mid- to long-chain carboxylic acids are isopropyl myristate and isopropyl stearate.

The ratio of the water-miscible to water-immiscible solvents in the organic carrier is determined based on the following criteria:

a). the resultant organic carrier is a substantially homogenous solution;
b). the active ingredient and organic polymer are substantially solubilized in the organic carrier;
c). the resultant organic carrier offers optimal absorption/penetration enhancement for the active ingredient since many water-miscible and water-immiscible solvents are also absorption/penetration enhancers;
d). the resultant organic carrier has favorable cosmetic properties and/or pharmacological safety profile.

The water-miscible solvent in the organic carrier may be a single solvent or a mixture of water-miscible solvents in any suitable ratio, for example, a mixture of ethanol and N-methyl-2-pyrrolidone, or a mixture of ethanol, propylene glycol, and N-methyl-2-pyrrolidone. The water-immiscible solvent in the organic carrier may be a single solvent or a mixture of water-immiscible solvents in any suitable ratio, for example, a mixture of capric/caprylic triglyceride and soy bean oil.

The ratio of the water-miscible to water-immiscible solvents in the organic carrier may be in the range of from 100:1 to 1:100 by weight, preferably from 50:1 to 1:50 by weight, more preferably from 10:1 to 1:10 by weight. The overall content of the organic carrier in the composition may be up to 70% by weight, preferably up to 50% by weight, more preferably up to 30% by weight.

The term, ‘affect efficacy’, ‘affected efficacy’, or ‘affecting efficacy’, in accordance with the present invention, means any, some, or all of the following definitions:

a. exerting cosmetic and/or pharmacological effect in a faster and/or more profound fashion;
b. modulating release profile of the active ingredient contained in the composition of the present invention to achieve desired cosmetic and/or pharmacological effect, such as, for example, to achieve a zero-order release profile, or pulsating release profile, or fast onset followed by zero-order release profile, or any desired type of release profile suitable for providing a desired cosmetic benefit or treating a particular medical condition;
c. providing rub-off resistance and/or water-proof properties so that the composition may be applied at lower and/or less frequent dosage, thereby reducing potential of side effects and/or adverse reactions;
d. reducing side effects such as skin irritation, allergy, or eczema, caused by direct contact with the active ingredient. The polymer may serve as an entrapping matrix for the active ingredient to minimize its direct contact with skin, thereby reducing potential of the side effects;
e. enhancing chemical stability of the active ingredient. The polymer may form a protective matrix around the active ingredient to reduce potential chemical degradation;
f. being able to formulate the composition with chemically incompatible ingredients (active or other ingredients) in the presence of the organic polymer where the polymer may form protective layers around the incompatible ingredients to prevent or minimize any degradative chemical reactions among them;
g. keeping the active ingredient in essentially amorphous form in the presence of the organic polymer. The solubility of active ingredient crystals in general is lower than for an amorphous active ingredient. It is also known that as the loading of an active ingredient in a composition is increased, the tendency for crystallization also increases. Thus, it is advantageous that the composition contains active ingredient in essentially amorphous form. A number of organic polymers are known crystallization inhibitors. For example, polyvinylpyrrolidone is a known and widely used crystallization inhibitor in pharmaceutical applications.

The organic polymer may be any cosmetically or pharmacologically acceptable polymer. In accordance with the present invention, the organic polymer is substantially solubilized in the organic carrier. The polymer may serve as a matrix for the active ingredient. The polymer matrix may affect efficacy of the active ingredient trapped in the matrix. The organic polymer according to the present invention may be a hydrophobic polymer, hydrophilic polymer, or any suitable combinations thereof. A hydrophobic polymer may be water soluble or insoluble. The polymer combinations may be any two or more hydrophobic polymers, two or more hydrophilic polymers, or a mixture of at least one hydrophobic polymer and at least one hydrophilic polymer, in suitable ratios for desired therapeutic efficacy, gelanic properties, or cosmetic benefits.

Selection of the organic polymer depends on nature of the active ingredient, organic carrier, and desired effect of the active ingredient. For example, a polymer with carboxylic group (e.g., acrylic acid) may interact with an active ingredient with basic group (e.g., the imidazole group found in many anti-fungal agents) through ionic interactions, potentially slowing down its release from the matrix. The ionic interactions between the active ingredient and polymer may provide a platform for a modulated release profile through a controlled ion-exchange or osmotic mechanism.

Examples of the hydrophobic polymers include, but not limited to: octylacrylamide acrylate copolymer, octylpropenamide acrylate copolymer, aminoalkyl methacrylate copolymer, polyvinyl alcohol, polyvinyl alcohol vinylacetate copolymer, polyvinylpyrrolidone vinylacetate copolymer, and suitable combinations thereof.

The copolymers may contain hydrophobic and hydrophilic monomer units. The copolymers may be random copolymers or block copolymers prepared through techniques such as grafting or living polymerization. The ratio of the hydrophobic to hydrophilic monomer units within the copolymer may be varied to adjust overall hydrophobicity/hydrophilicity of the copolymer. For example, octylacrylamide is a hydrophobic monomer unit. On the other hand, acrylate, in acid or salt form, is a hydrophilic monomer unit. Higher octylacrylamide to acrylate ratio gives more hydrophobic octylacrylamide acrylate copolymer. Vinylpyrrolidone is a hydrophilic monomer unit. Thus, higher concentration of hydrophobic vinyl acetate gives more hydrophobic polyvinylpyrrolidone vinylacetate copolymer. Polyvinyl alcohol is obtained from complete hydrolysis of polyvinylacetate. Partial hydrolysis of polyvinylacetate produces a copolymer of polyvinyl alcohol vinylacetate. Degree of the hydrolysis determines the hydrophobicity/hydrophilicity of the copolymer.

This variation in hydrophobicity/hydrophilicity in the copolymers may affect interactions between the active ingredients and copolymers in a systematically controlled fashion, thereby affecting efficacy of the compositions.

In addition, the polymer with higher hydrophobicity generally has lower water solubility, which makes the composition containing the polymer more water-proof and rub-off resistant.

There are in general two ways to modulate hydrophobicity/hydrophilicity of the polymer contained in the composition in accordance with the present invention. One is to use a mixture of hydrophobic and hydrophilic polymers at various ratios to control overall hydrophobicity/hydrophilicity of the polymer matrix. The other is to use a copolymer and control hydrophobicity/hydrophilicity of the copolymer by varying ratios of the hydrophobic to hydrophilic monomer units in the copolymer.

Examples of the hydrophilic polymers include, but not limited to: hydroxyalkyl cellulose, polyvinylpyrrolidone, polylactide, polyglycolide, polycaprolactone, or copolymers thereof, and suitable combinations thereof. Examples of the hydroxyalkyl cellulose are hydroxyethyl cellulose, hydroxypropyl cellulose, and hydroxypropyl methylcellulose.

Overall concentration of the polymer in the organic carrier may be up to 50% by weight, preferably up to 30% by weight, more preferably up to 20% by weight. When a mixture of polymers is used, concentration of each polymer may be in the range of from 0.01% to 99.99% by weight. The ratio among the polymers is determined by cosmetic and/or efficacy requirement. For a mixture of two polymers, for example, hydroxypropyl cellulose and octylacrylamide acrylate copolymer, the ratio may be in the range of from 100:1 to 1:100 by weight, preferably from 10:1 to 1:10 by weight. For a mixture of three polymers, for example, hydroxypropyl cellulose, polyvinylpyrrolidone vinylacetate copolymer, and octylacrylamide acrylate copolymer, the ratio may be in the range of from 100:1:1 to 1:100:1 to 1:1:100 by weight, preferably from 10:1:1 to 1:10:1 to 1:1:10 by weight. For mixtures of four or more polymers, concentration of each polymer and ratio among the polymers follow similar pattern as discussed above.

The overall content of the polymer in the composition may be up to 40% by weight, preferably up to 30% by weight, more preferably up to 20% by weight.

The active ingredient may be any suitable chemical compound. The active ingredient may be a cosmetic or pharmacologically active agent. The active ingredient may include cosmetic or pharmacologically active agents that have not been discovered yet.

All physical forms of a given active agent, crystalline, semi-crystalline, and amorphous, are contemplated and within the scope of the present invention, either in admixture or in pure or substantially pure form. Furthermore, the stereoisomers of a given active agent are also contemplated and within the scope of the present invention. The definition of a given active agent in accordance with the present invention embraces all possible stereoisomers and their mixtures. It particularly embraces the racemic forms and the isolated optical isomers having the specified activity.

Examples of the cosmetic agents include, but not limited to:

Water-immiscible vitamins, for example, tocopherols (vitamin E), tocopherol derivatives, tocotrienols, vitamin D, K and derivatives thereof, and suitable combinations thereof;

Moisturizing agents, for example, petrolatum, squalane, hydrogenated polydecene, isododecane, isohexadecane, mineral oils, vegetable oils and waxes, synthetic waxes, emollient esters, lanolins and their derivatives, unsaturated fatty acids and their derivatives, and suitable combinations thereof;

Water-immiscible antioxidants, for example, propyl gallate, butylated hydroxytoluene, vitamin C palmitate, butylated hydroxyanisole, carotenoids, coenzymes Q (e.g., co-enzyme Q-10), and lipoic acid, and suitable combinations thereof;

Sun protection chemicals, for example, 2-ethylhexyl-3-cyano-3,3-diphenyl-2-propenoate (octocrylene), butyl methoxydibenzoylmethane (avobenzene), 2-ethylhexyl methoxycinnamate (octyl methoxycinnamate), oxybenzone (benzophenone-3), sulisobenzone (benzophenone-4), dioxybenzone (benzophenone-8), and 2-ethylhexyl salicylate (octyl salicylate), and suitable combinations thereof.

Suitable combinations of the cosmetic agents may be used in the compositions of the present invention as long as such combinations offer cosmetic benefits.

Examples of the pharmacologically active agents include, but not limited to:

Cardioactive agents, for example, organic nitrates such as nitroglycerine, isosorbide dinitrate, and isosorbide mononitrates; quinidine sulfate; procainamide; thiazides such as chlorothiazide, and hydrochlorothiazide; nifendipine, nicardipine, adrenergic blockers, such as timolol, propranolol, verapamil, dithiazem, captopril, and clonidine;

Estrogens, for example, conjugated estrogens, esterified estrogens, 17-beta-estradiol, 17-beta-estradiol valerate, mestranol, estriol, or any estrogen or its derivatives suitable for use in hormone replacement therapy;

Retinoids, a “retinoid” is a keratolytic drug related to retinoic acid and generally includes chemical entities such as retinol and its esters and closely related naturally-occurring derivatives and structurally-related synthetic analogs. This includes, for example, retinol, retinal, tretinoin (all-trans retinoic acid), isotretinoin, adapalene (6-[3-(1-adamantyl)-4-methoxyphenyl]-2-naphthoic acid), and the like. Of these, tretinoin is preferred;

Analgesics and anesthetics, for example, lidocaine, tetracaine, dibucaine, prilocalne, benzocaine, capsaicin, and fentanyl;

Corticosteroid family of anti-inflammatory, anti-pruitic agents, for example, hydrocortisone, cortisone, desonide, clobetasol, dexmethasone, fluocinolone, fluocinonide, prednisolone, diflorasone, betamethasone, and derivatives thereof;

Non-steroid family of anti-inflammatory agents, for example, ibuprofen, diclofenac, naproxen, and methyl salicylate;

Anti-viral agents, for example, acyclovir, and the like;

Anti-microbial agents, for example, benzoyl peroxide, mupirocin, cephalosporins, and penicillins;

Anti-fungal agents, for example, clotrimazole, ketoconazole, miconazole, itraconazole, and fluconazole;

Skin cancer treatment agent, for example, fluorouracil;

Rosacea treatment agent, for example, metronidazole;

Hair growth agent, for example, minoxidil.

Suitable combinations of the pharmacologically active agents may be used in the compositions of the present invention as long as such combinations offer pharmacological advantages for treating a medical condition.

Additional examples of pharmacologically active agents include those compounds which are substantially water insoluble and which are listed in the “Therapeutic Category and Biological Activity Index” of the Merck Index (12^{th} edition, 1996), the entire contents of which are thereby incorporated by reference.

The amount of the active ingredient in the composition is a safe and effective amount for that compound. The term ‘safe and effective amount’, as used herein, means an amount of an active ingredient used in the compositions and methods of the present invention, sufficient enough to significantly and positively modify the condition to be treated but low enough to avoid serious side effects, within the scope of sound medical advice.

The compositions of the present invention may include one or more skin absorption/penetration enhancers which enhance the absorption and/or penetration of the active ingredient. The absorption/penetration enhancers may be water-miscible or water-immiscible. The absorption/penetration enhancers may be any suitable enhancers in the art. For example, many water-miscible and water-immiscible solvents are absorption/penetration enhancers. The organic carrier in accordance with the present invention may also function as the absorption/penetration enhancers in addition to its role as a carrier for the active ingredient and organic polymer. Additional absorption/penetration enhancers may be included in the compositions as well. The absorption/penetration enhancers may be included in the aqueous or organic phase.

The aqueous gel is here defined as a dimensionally stable hydrogel which is formed by processing one or several gelling agents (often referred to as thickening agents or thickeners) with water. In accordance with the present invention, the aqueous gel phase consists essentially of water and the gelling agents. The aqueous gel phase may also contain limited amounts of water-miscible solvents, for example, ethanol, glycerin, propylene glycol, and PEGs. Examples of the gelling agents include, but not limited to, agar, xanthan gums, cellulose, alginates, synthetic derivatives of cellulose, and acrylic polymers. Examples of synthetic derivatives of cellulose are hydroxyalkyl cellulose such as hydroxyethyl cellulose and hydroxypropyl cellulose. Examples of acrylic polymers are a family of thickening agents known under tradename Carbopol, such as Carbopol 934, 940, 941, Ultrez 10, and Ultrez 20.

The term, ‘free of surface-active substances’, as used herein, means that the composition in accordance with the present invention does not contain conventional surface-active substances, such as, for example, cationic, anionic or nonionic surfactants or emulsifiers. The gelling agents with surface-active properties are tolerable as long as they do not impair the major galenic benefits of the topical compositions within the scope of the present invention. For example, Carbopol Ultrez 20 contains C.sub.10-30 alkyl groups which have surface-active properties.

The organic solution containing at least one solubilized active ingredient and at least one solubilized organic polymer is worked into the aqueous gel by mixing until desired degree of dispersion is achieved. The degree of dispersion is largely determined by selection of the dispersing tool. High-speed/high pressure tools (e.g., homogenizers) yield smaller particle sizes while simple mechanical mixers (e.g., blade mixers) yield larger particle sizes.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

Preparation of Topical Composition with Model Drug—An Anti-Fungal Agent

This example describes a composition containing clotrimazole (an anti-fungal agent) as active ingredient and a mixture of a hydrophobic polymer, polyvinylpyrrolidone vinylacetate copolymer (PVP/VA) and hydrophilic polymer, hydroxypropylcellulose (HPC):

| Component | Amount (weight percentage) |
|---|---|
| Phase A: | |
| Clotrimazole | 1.0% |
| PVP/VA | 2.0% |
| HPC | 2.0% |
| Capric/caprylic triglyceride | 5.0% |
| Ethanol | 15.0% |
| Phase B: | |
| Water | 74.50% |
| Carbopol Ultrez 20 | 0.5% |

Phase A was prepared by dissolving PVP/VA (sold under tradename Plasdone S-630) and HPC (sold under tradename Klucel) in ethanol. Then the triglyceride was added to the solution to yield a solution. Clotrimazole was added to the solution and dissolved. Phase B was prepared by shifting Ultrez 20 into water. After Ultrez 20 was wetted, triethanol amine (TEA) was added to adjust pH to about 6.5 to 7.0 to form an aqueous gel. Phase A was worked into Phase B by mechanical mixing. A white cream was formed.

### EXAMPLE 2

Preparation of Topical Composition with Model Drug—A Corticosteroid

This example describes a composition containing hydrocortisone 21-acetate (a corticosteroid) as active ingredient and a hydrophobic, water-insoluble polymer, octylacrylamide acrylate copolymer (sold under tradename Dermacryl 79):

| Component | Amount (weight percentage) |
|---|---|
| Phase A: | |
| Hydrocortisone acetate | 1.0% |
| Dermacryl 79 | 1.0% |
| Capric/caprylic triglyceride | 5.0% |
| N-Methyl-2-pyrrolidone (NMP) | 10.0% |
| Phase B: | |
| Water | 82.50% |
| Carbopol Ultrez 20 | 0.5% |

Phase A was prepared by dissolving Dermacryl 79 polymer in NMP. Then the triglyceride was added to the solution and solubilized. Hydrocortisone acetate was added to the solution and dissolved. Phase B was prepared by shifting Ultrez 20 into water. After Ultrez 20 was wetted, triethanol amine (TEA) was added to adjust pH to about 5.5 to 6.0 to form an aqueous gel. Phase A was worked into Phase B by mechanical mixing. A white cream was formed.

### EXAMPLE 3

Preparation of Topical Composition with Model Drug—A Corticosteroid

This example describes a composition containing hydrocortisone 21-acetate (a corticosteroid) as active ingredient and a hydrophobic, water-soluble polymer, PVP/VA:

| Component | Amount (weight percentage) |
|---|---|
| Phase A: | |
| Hydrocortisone acetate | 1.0% |
| PVP/VA | 2.5% |
| Capric/caprylic triglyceride | 5.0% |
| N-Methyl-2-pyrrolidone (NMP) | 10.0% |
| Phase B: | |
| Water | 81.0% |
| Carbopol Ultrez 10 | 0.5% |

Phase A was prepared by dissolving PVP/VA polymer in NMP. Then the triglyceride was added to the solution and solubilized. Hydrocortisone acetate was added to the solution and dissolved to form a slightly hazy solution. Phase B was prepared by shifting Ultrez 10 into water. After Ultrez 10 was wetted, triethanol amine (TEA) was added to adjust pH to about 5.5 to 6.0 to form an aqueous gel. Phase A was worked into Phase B by mechanical mixing. A white cream was formed.

### EXAMPLE 4 (FOR COMPARISON WITH EXAMPLE 3)

Preparation of Topical Composition with Model Drug—A Corticosteroid without a Polymer

This example describes a composition containing hydrocortisone 21-acetate (a corticosteroid) as active ingredient without a polymer:

| Component | Amount (weight percentage) |
|---|---|
| Phase A: | |
| Hydrocortisone acetate | 1.0% |
| Capric/caprylic triglyceride | 5.0% |
| N-Methyl-2-pyrrolidone (NMP) | 10.0% |
| Phase B: | |
| Water | 83.5% |
| Carbopol Ultrez 10 | 0.5% |

Phase A was prepared by hydrocortisone acetate in NMP. Then the triglyceride was added to the solution and solubilized to form a clear solution. Phase B was prepared by shifting Ultrez 10 into water. After Ultrez 10 was wetted, triethanol amine (TEA) was added to adjust pH to about 5.5 to 6.0 to form an aqueous gel. Phase A was worked into Phase B by mechanical mixing. A white cream was formed.

### EXAMPLE 5

Preparation of Topical Composition with Model Drug—A Local Anesthetic

This example describes a composition containing lidocaine (an anesthetic) as active ingredient and a hydrophobic, water-soluble polymer, PVP/VA:

| Component | Amount (weight percentage) |
|---|---|
| Phase A: | |
| Lidocaine | 5.0% |
| PVP/VA | 3.0% |
| Capric/caprylic triglyceride | 5.0% |
| NMP | 15.0% |
| Phase B: | |
| Water | 71.50% |
| Carbopol Ultrez 20 | 0.5% |

Phase A was prepared by dissolving PVP/VA in NMP. Then the triglyceride was added to the solution. Lidocaine was added to the solution and dissolved. Phase B was prepared by shifting Ultrez 20 into water. After Ultrez 20 was wetted, triethanol amine (TEA) was added to adjust pH to about 7.0 to 7.5 to form an aqueous gel. Phase A was worked into Phase B by mechanical mixing. A white cream was formed.

### EXAMPLE 6 (FOR COMPARISON WITH EXAMPLE 5)

Preparation of Topical Composition with Model Drug—A Local Anesthetic without a Polymer

| Component | Amount (weight percentage) |
|---|---|
| Phase A: | |
| Lidocaine | 5.0% |
| Capric/caprylic triglyceride | 5.0% |
| NMP | 15.0% |
| Phase B: | |
| Water | 74.50% |
| Carbopol Ultrez 20 | 0.5% |

Phase A was prepared by dissolving lidocaine in NMP. Then the triglyceride was added to the solution. Phase B was prepared by shifting Ultrez 20 into water. After Ultrez 20 was wetted, triethanol amine (TEA) was added to adjust pH to about 7.0 to 7.5 to form an aqueous gel. Phase A was worked into Phase B by mechanical mixing. A white cream was formed.

### EXAMPLE 7

Preparation of Topical Composition with Model Drug—A Local Anesthetic Using Polysaccharide Based Gelling Agent

This example describes a composition containing lidocaine (an anesthetic) as active ingredient using a polysaccharide based gelling agent:

| Component | Amount (weight percentage) |
|---|---|
| Phase A: | |
| Lidocaine | 5.0% |
| PVP/VA | 3.0% |
| Capric/caprylic triglyceride | 5.0% |
| NMP | 15.0% |
| Phase B: | |
| Water | 70.50% |
| Xanthan gum | 1.5% |

Phase A was prepared by dissolving PVP/VA in NMP. Then the triglyceride was added to the solution. Lidocaine was added to the solution and dissolved. Phase B was prepared by dispersing xanthan gum into water in a shifted fashion. The mixture was stirred until an aqueous gel was formed. Phase A was worked into Phase B by mechanical mixing. A white cream was formed.

### EXAMPLE 8

### Comparison of the Therapeutic Efficacy—Anti-Inflammatory Effect

This example is to compare anti-inflammatory efficacy of hydrocortisone acetate in the compositions prepared in Examples 3 and 4 and demonstrate effect of the polymer on efficacy of the composition.

Topical corticosteroids share their anti-inflammatory, anti-pruitic, and vasoconstrictive actions. The ability of a given corticosteroid agent to cause vasoconstriction usually correlates with its anti-inflammatory potency. Vasoconstrictor assays are used in the art and by the U.S. Food and Drug Administration for determining the potency of topical corticosteroid preparations.

Vasoconstrictor assay was used to evaluate and compare the two compositions. The compositions were applied to two areas of volar forearm of a human subject. The application sites were about 1.5 (width)×2 cm (length) bands in parallel and their edges were approximately 4 cm apart. About 20 mg of each product was applied. The product was evenly spread over the entire site using a glass rod. After the application sites were dried, a raised plastic guard was used to protect each application site. At 17 hours after application, the protective covers were removed. The sites were cleaned by washing with mild soap and warm water. Skin vasoconstrictor evaluations were performed on a point scale (0-4) at about 19 hours. Three observers were blinded with respect to the compositions used in the assay. The point scale scores were defined as follows:

Point score scale: 0=normal skin; 1=slight blanching of indistinct outline; 2=more intense blanching; 3=even blanching with a clear outline of the application site; and 4=intense blanching.

| | Example 3 | Example 4 |
|---|---|---|
| Blanching score | 1-2 | 0-1 |

All three observers could independently tell difference in the skin color between the two application sites. The difference in the skin color between the application sites and the surrounding skin was also observable.

This result clearly suggests that the composition containing the organic polymer shows affected therapeutic (anti-inflammatory) efficacy.

### EXAMPLE 9

### Comparison of the Therapeutic Efficacy—Local Anesthetic Effect

This example is to compare local anesthetic efficacy of the compositions prepared in Examples 5 and 6 and demonstrate effect of the polymer on efficacy of the composition.

The compositions were applied to two areas of volar forearm of a human subject. The application sites were circles of about 2 cm in diameter and their centers were approximately 5 cm apart. At predetermined time intervals, the skin area being treated were pricked with a blunt needle and the pain sensation was recorded. The effect scores were defined as follows:

Pain score scale: 0=no effect, 1=no effect to medium numb, 2=medium numb, 3=almost completely numb, 4=completely numb, 5=completely numb and deep.

| Time (min) | Example 5 | Example 6 |
|---|---|---|
| 0 | 0 | 0 |
| 30 | 0 | 0 |
| 60 | 1 | 0 |
| 75 | 1-2 | 0-1 |
| 90 | 2-3 | 1 |
| 105 | 3 | 1-2 |
| 120 | 3-4 | 2 |
| 135 | 4 | 2 |
| 150 | 4-5 | 2 |

This result clearly suggests shorter onset time and higher local anesthetic efficacy for the composition containing the organic polymer.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A topical composition in the form of a dispersion gel comprising:
at least one aqueous gel phase formed by processing at least one gelling agent with water,
at least one organic phase formed by dispersing into the aqueous gel phase a substantially homogenous solution comprising at least one active ingredient and at least one organic polymer solubilized in an organic carrier consisting essentially of at least one water-miscible organic solvent and at least one water-immiscible organic solvent,
wherein the organic polymer is selected to affect efficacy of the composition and the composition is essentially free of surface-active substances.

2. The composition in claim 1 wherein the organic polymer is selected from the group consisting of a hydrophilic polymer, hydrophobic polymer, and combinations thereof.

3. The composition in claim 2 wherein the hydrophilic polymer is selected from the group consisting of hydroxypropyl cellulose, hydroxypropyl methylcellulose, hydroxyethyl cellulose, polyvinylpyrrolidone, polylactide, polyglycolide, polycaprolactone, or copolymers thereof, and combinations thereof.

4. The composition in claim 2 wherein the hydrophobic polymer is selected from the group consisting of octylacrylamide acrylate copolymer, octylpropenamide acrylate copolymer, aminoalkyl methacrylate copolymer, polyvinyl alcohol, polyvinyl alcohol vinylacetate copolymer, polyvinylpyrrolidone vinylacetate copolymer, and combinations thereof.

5. The composition in claim 1 wherein the organic polymer is a copolymer consisting essentially of hydrophobic and hydrophilic monomer units.

6. The composition in claim 1 wherein the organic polymer is a mixture of at least one hydrophilic polymer and at least one hydrophobic polymer.

7. The composition in claim 1 wherein said active ingredient is a cosmetic agent.

8. The composition in claim 1 wherein said active ingredient is a pharmacologically active agent.

9. The composition in claim 8 wherein the pharmacologically active agent is selected from the group consisting of an anti-inflammatory agent, anti-pruitic agent, anti-fungal agent, anti-microbial, analgesic agent, estrogen, a cardioactive agent, and retinoid.

10. The composition in claim 7 wherein the cosmetic agent is selected from the group consisting of a water-immiscible vitamin, moisturizing agent, water-immiscible antioxidant, sun protection chemical, and combinations thereof.

11. A composition in the form of a dispersion gel for topical delivery of at least one active ingredient, comprising:
at least one aqueous gel phase formed by processing at least one gelling agent with water,
at least one organic phase formed by dispersing into the aqueous gel phase a substantially homogenous solution comprising said active ingredient and at least one organic polymer solubilized in an organic carrier consisting essentially of at least one water-miscible organic solvent and at least one water-immiscible organic solvent,
wherein the organic polymer is selected to affect efficacy of the composition and the composition is essentially free of surface-active substances.

12. The composition in claim 11 wherein the organic polymer is selected from the group consisting of a hydrophilic polymer, hydrophobic polymer, and combinations thereof.

13. The composition in claim 11 wherein the organic polymer is a mixture of at least one hydrophilic polymer and at least one hydrophobic polymer.

14. The composition in claim 11 wherein the organic polymer is a copolymer consisting essentially of hydrophobic and hydrophilic monomer units.

15. The composition in claim 12 wherein the hydrophilic polymer is selected from the group consisting of hydroxypropyl cellulose, hydroxypropyl methylcellulose, hydroxyethyl cellulose, polyvinylpyrrolidone, polylactide, polyglycolide, polycaprolactone, or copolymers thereof, and combinations thereof.

16. The composition in claim 12 wherein the hydrophobic polymer is selected from the group consisting of octylacrylamide acrylate copolymer, octylpropenamide acrylate copolymer, aminoalkyl methacrylate copolymer, polyvinyl alcohol, polyvinyl alcohol vinylacetate copolymer, polyvinylpyrrolidone vinylacetate copolymer, and combinations thereof.

17. The composition in claim 11 wherein said active ingredient is a cosmetic agent.

18. The composition in claim 11 wherein said active ingredient is a pharmacologically active agent.

19. The composition in claim 18 wherein the pharmacologically active agent is selected from the group consisting of an anti-inflammatory agent, anti-pruitic agent, anti-fungal agent, anti-microbial, analgesic agent, estrogen, a cardioactive agent, and retinoid.

20. A method of prophylactic or therapeutic anti-fungal, anti-microbial, estrogen, keratolytic, analgesic, anti-inflammatory, or anti-pruitic treatment including administering to a patient in need of such treatment a safe and effective amount of the composition as claimed in claim 1.
